(19) United States
(12) Patent Application Publication
(10) Pub. No.: US 2012/0046523 A1
(43) Pub. Date: Feb. 23, 2012

(54) SELF PROPELLING DEVICE

(76) Inventors: Shinichi YAMAKAWA, Kanagawa (JP); Takayuki Nakamura, Kanagawa (JP); Masayuki Iwasaka, Kanagawa (JP)

(21) Appl. No.: 13/213,891

(22) Filed: Aug. 19, 2011

(30) Foreign Application Priority Data
Aug. 20, 2010 (JP) 2010-185193

(51) Int. Cl. A61B 1/00

(52) U.S. Cl. 600/118

(57) ABSTRACT

A self propelling device is attached to a front end side of an insert section of an endoscope and propels the front end side in a body. The self propelling device has an internal cylinder into which the front end side is inserted and an external cylinder surrounding the internal cylinder. A rotating body is rotatably attached to the external cylinder in an axis direction along the inner periphery and the outer periphery of the external cylinder, and the self propelling device propels by rotation of the rotating body. The inner diameter of the internal cylinder is large, and the front end side can be bendable in the internal cylinder. A coil spring formed in a circular truncated cone shape is located in the internal cylinder. The coil spring has a bottom having large diameter which is fixed to the internal cylinder and has a top having small diameter which is fit on the front end side of the insert section. The self propelling device and the front end side of the insert section are connected via the coil spring so that they can relatively move.

### FIELD OF THE INVENTION

This present invention relates to a self propelling device for propelling an insert section of an endoscope in a body.

### BACKGROUND OF THE INVENTION

An endoscope is provided with an insert section inserted into a body and an operation unit for operating the insert section. The insert section is sectioned into a distal portion, a bending portion and flexible portion. The distal portion and the bending portion are located at a front end side of the insert section. For example, an imaging capturing window for importing a target image is located on a front surface of the distal portion. The bending portion bends by operation of the operation unit to orient the distal portion toward a desired direction. The flexible portion has a length approximately 1 m to 2 m depending on the intended purpose of the endoscope.

In a colonoscope, the insert section is inserted into a large intestine from an anus. In the large intestine, since a sigmoid colon and a transverse colon relatively freely move in the body, it is difficult to advance the insert section deeply in the sigmoid colon or transverse colon, and much experience of manipulation of the endoscope is required. If a doctor is insufficiently skilled in the manipulation, physical load to the body of a patient will be very large. In view of such a problem, in order to assist the insertion, a self propelling device which propels the insert section in a body, for example, in a body cavity has been developed in recent years.

JP-A 2009-195321 discloses a self propelling device for inserting the insert section into a deep part in the large intestine, for example, back of a SD junction that is a boundary between the sigmoid colon and transverse colon. The self propelling device is provided with an extensible unit attached to a front end side of the inserter, and a balloon that is attached to a rear end of the extensible unit. While the balloon swells and contacts an intestine wall at the front of the SD junction, the extensible unit forwardly extends and the front end side of the insert section goes over the SD junction to advance deeply. Thereby, the insert section can be inserted into the back of the SD junction.

The self-propelling device disclosed in U.S. 2006/0089533 (corresponding to JP-A 2009-513250) is provided with an internal cylinder attached to the front end side of the inserter, and an external cylinder positioned outside of the internal cylinder, and a rotating body. The rotating body is wound between an inner periphery and an outer periphery of the external cylinder. The rotating body is supported by the external cylinder so as to rotate along an axial direction. The insert section can be inserted into the deep part of the large intestine by rotation of the rotating body in a manner that the rotating body contacts the intestine wall of the large intestine.

The self propelling device disclosed in the JP-A 2009-195321 is constituted of the extensible unit and the balloon, so that the self propelling device is formed long in an axial direction of the insert section. Thereby, the entire of the bending portion is covered with the self propelling device, in addition to the part of the distal portion and the part of the flexible portion. Therefore, there is a possibility that the bending operation, for example, bending of the bending portion at 180° is blocked by the self propelling device.

Also in the self propelling device disclosed in the WO 2007/050370, the internal cylinder and the external cylinder are formed long in the axial direction of the insert section, so that the entire of the bending portion is covered with the self propelling device. Therefore, problems arise in that the bending operation of the bending portion is blocked and the flexibility of the flexible portion is partly lost due to the self propelling device. As a result, the insert technique becomes more difficult.

### SUMMARY OF THE INVENTION

An object of the present invention is to provide a self propelling device for allowing smooth bending operation of a front end side of an insert section.

In order to achieve the above object, a self propelling device of the present invention has an internal cylinder, an external cylinder, a rotating body, a driver and a support member. The self propelling device is attached to a bendable front end side of an insert section of an endoscope and propels the front end side in a body. The internal cylinder has an insertion hole into which the front end side is inserted with a predetermined space. The external cylinder is disposed outside of the internal cylinder. The rotating body is wound to surround the outer periphery and inner periphery of the external cylinder. The driver rotates the rotating body along the axis direction of the external cylinder. The support member is provided between an inner periphery of the internal cylinder and an outer periphery of the front end side, and movably supports the front end side in an at least radial direction in the insertion hole. The driver rotates the rotating body to propel the front end side in the body.

A first end of the support member is preferably fixed to the inner periphery of the internal cylinder, and a second end of the support member is detachably fit onto the outer periphery of the front end side, and the first end and the second end are separated in the axis direction. Moreover, the support member is preferably a coil spring formed in a circular truncated cone shape, and the first end is positioned at a bottom having large diameter and the second end is positioned at a top having small diameter. Furthermore, the second end is preferably fit onto an outer periphery of a bending portion that is bendable and provided at the front end side. Still furthermore, the internal cylinder and the external cylinder are preferably formed in skirt shape gradually spreading toward the rear end side of the insert section.

According to the present inversion, since the front end of the insert section is movably supported in an at least radial direction in the insert section, it is possible to prevent the self propelling device from blocking bending operation of the front end when the insert section is bended. Thereby, the bending operation of the front end side can be smoothly carried out.

Moreover, one end of the support member and the other end thereof are separated along an axis direction of the internal cylinder, so that the front end side is easy to move in the radial direction in the insertion hole when the front end side is bended. Therefore, the front end side can be easily bended in the insertion hole. As a result, further smooth bending operation of the front end side can be achieved.

### BRIEF DESCRIPTION OF THE DRAWINGS

The above objects and advantages of the present invention will become more apparent from the following detailed description when read in connection with the accompanying drawings, in which:

FIG. 1 is a schematic diagram showing an endoscope system;

FIG. 2 is a sectional view illustrating a manner that a self propelling device is attached to an insert section of an electronic endoscope according to a first embodiment of the present invention;

FIG. 3 is an explanatory view illustrating a size relation between the self propelling device and the insert section;

FIG. 4 is a sectional view illustrating an example that a coil spring is reversely disposed in a front-back direction; and

FIG. 5 is a sectional view illustrating a self propelling device according to a second embodiment.

### DESCRIPTION OF THE PREFERRED EMBODIMENTS

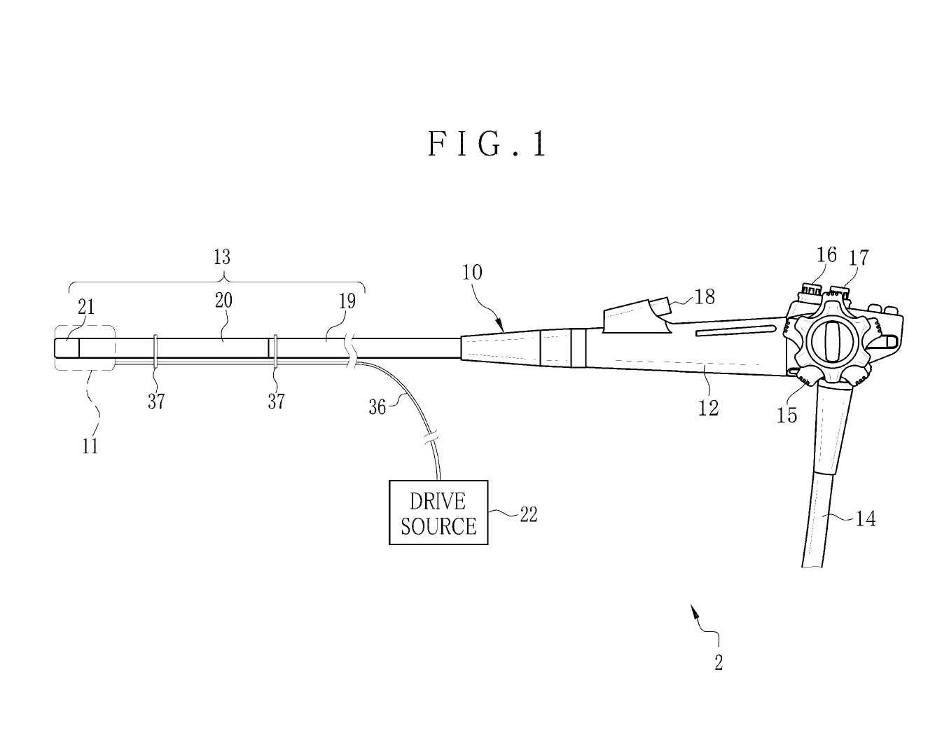

Shown in FIG. 1, an endoscope system 2 includes an electronic endoscope 10 and a self propelling device 11. The electronic endoscope 10 includes a handling section 12 and an insert section 13 that is connected to the handling section 12 and inserted into a large intestine in a body. A universal code 14 is connected to the handling section 12. Connecters (not shown) for respectively connecting to a light source device (not shown) and a processor (not shown) are provided at a front end of the universal code 14.

The handling section 12 is provided with an angle knob 15, an air/water feed button 16 and a suction button 17 and so on. Moreover, the handling section 12 is provided with a forceps channel 18 into which a medical instruments such as forceps and an electrosurgical knife are inserted.

The insert section 13 includes a flexible portion 19 having flexibility, a bending portion 20 that is freely bendable and a distal portion 21 in a sequence from the control handle 12. The bending portion 20 and the distal portion 21 constitute a front end side of the insert section.

The flexible portion 19 has a length, for example, 1.6 m for reach of the distal portion 21 to an object of interest in the large intestine. The bending portion 20 has a length, for example 10 cm to 13 cm, and bends up and down or right and left in response to operation of the angle knob 15. Thus, the distal portion 21 can be oriented in a desired direction.

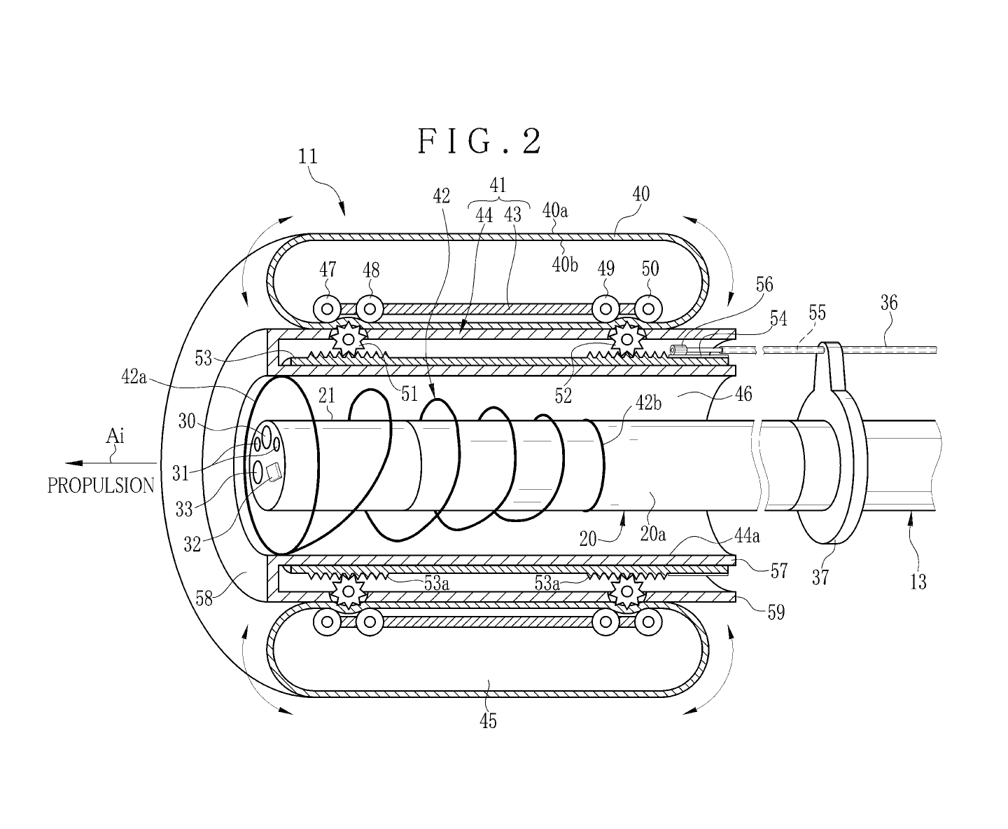

The distal portion 21 has a length about 15 mm, and includes an image capturing window 30 (see FIG. 2) for importing a target image in the large intestine. Objective optical system and image pickup device such as CCD or CMOS image sensors for capturing image of the target are provided at the back of the image capturing window 30. The image pickup device is connected to the processor by a signal cable that is accommodated in the insert section 13, the handling section 12 and the universal code 14. The processor executes image processing of various functions to the image signal obtained from the image pickup device through the signal cable to generate video signal and outputs the data on a monitor (not shown) connected through the cable. Then, an observation image of the target is displayed on the monitor.

Moreover, the distal portion 21 is provided with an illumination window 31, an air/water feed nozzle 32 and a forceps outlet port 33 and so on (see FIG. 2). The illumination window 31 illuminates illumination light that is led from the light source device through light fiber to the internal body part. The air/water feed nozzle 32 jets air or water supplied from air/water feed device (not shown) toward the image capturing window 30 when the air/water feed button 16 is pressed. The forceps outlet port 33 is an outlet of the front end of the medical instrument that is inserted through the insert section 13 from the forceps channel 18.

The self propelling device 11 is attached to the front end side of the insert section 13, and carries the insert section 13 forward and backward in the large intestine. The self propelling device 11 is driven by a drive source 22. The drive source 22 generates rotating torque for driving the self propelling device 11. The rotating torque is transmitted to the self propelling device 11 through a rotating torque transmission member 55 (see FIG. 2) connected to the drive source 22.

The rotating torque transmission member 55 is inserted through a protective sheath 36 formed from such as fluorine resin. The protective sheath 36 is supported by plural support rings 37 fixed onto outer periphery of the insert section 13. Therefore, the rotating torque transmission member 55 is supported along an axial direction of the insert section 13. A torque wire or torque coil is used as the rotating torque transmission member 55.

The drive source 22 is controlled by a controller (not shown). An operation unit (not shown) is attached to the controller. The operation unit includes a button for inputting command for moving forward, moving backward and stopping the self propelling device 11, and a speed change button for changing a moving speed. Note that it may be possible that a program corresponding to an observation object is prepared in advance, and the drive source 22 is driven according to the program to automatically actuate the self propelling device 11.

In FIG. 2, the self propelling device 11 includes a rotating body (rotor or toroid) 40, a main body 41 that is almost cylinder shape and a coil spring 42 for attaching the main body 41 to the insert section 13. The main body 41 includes an external cylinder 43 and an internal cylinder 44 that is placed inside of the external cylinder 43.

The rotating body 40 is wound between the outer periphery and inner periphery of the external cylinder 43 so as to wrap the entire of the external cylinder 43, and rotatably supported by the external cylinder 43. The rotating body 40 is formed from a material having flexibility, in particular, a biocompatible plastic material, such as polyvinyl chloride, polyamide resin, fluorocarbon resin and the like. The rotating body 40 is a hollow structure having an outer surface 40a and an inner surface 40b. The inner part of the rotating body 40 is filled with anyone of fluid such as gas and liquid, or combination thereof.

The rotating body 40 rotates along an axial direction of the internal cylinder 44 in a manner that the outer surface 40a contacts the inner wall of the large intestine, shown with an allow line. Thereby, the rotating body 40 generates propulsion force for carrying the insert section 13 forward and backward along an insert direction Ai.

For example, when the insert section 13 is carried forward in the insert direction Ai, the rotating body 40 that contacts inner wall of the large intestine moves in a pulling direction reverse to the insert direction Ai, and turns at 180° at the rear end of the external cylinder 43 to be folded inside of the external cylinder 43. After the rotating body 40 moves in the insert direction Ai in the inside of the external cylinder 43, the rotating body 40 turns at 180° at the front end of the external cylinder 43 to be folded outside of the external cylinder 43. While the outside of The rotating body 40 moves along the pulling direction, the inside of the rotating body 40 moves along the insert direction Ai to carry the insert section 13 forward. On the other hand, when the insert section 13 is carried backward in the pulling direction, the outside of the rotating body 40 rotates so as to move the outside thereof along the insert direction Ai and move the inside thereof in the purring direction.

The external cylinder 43 is positioned in a space 45 surrounded by the rotating body 40 to support the rotating body 40. The internal cylinder 44 is disposed between the insert section 13 and the rotating body 40. The internal cylinder 44 has an insertion hole 44a through which the bending portion 20 and the distal portion 21 (hereinafter referred to as the bending portion 20 et al.) are inserted. The inner diameter of the insertion hole 44a is sufficiently larger than diameter of the bending portion 20 et al. Thereby, a space 46 is formed in the insertion hole 44a for smooth bending of the bending portion 20.

The coil spring 42 is formed in truncated cone shape, in which one end is positioned at a bottom and the other end is positioned at a top. A large loop (annular part) 42a having large diameter is located at the bottom and a small loop (annular part) 42b having small diameter is located at the top. The whole circumference of the large loop 42a is fixed onto the inner periphery of the internal cylinder 44 forming the insertion hole 44a. The whole circumference of the small loop 42b detachably engages with the outer periphery 20a of the bending portion 20. Thereby, the bending portion 20 et al. are supported in the air in the insertion hole 44a. The front end side is movable and rotatable in an axis direction relative to the self propelling device 11 in the range of elastic deformation of the coil spring 42.

It is noted that position for fixing the large loop 42a to the inner periphery of the internal cylinder 44 and the position for fixing the small loop 42b to the outer periphery 20a are separated each other in an axis direction of the internal cylinder 44. This allows easy bending of the bending portion 20 upon bending the bending portion 20, compared to the situation the both fixing positions are not separated.

Driven rollers 47 to 50 are rotatably attached to the periphery of the external cylinder 43 along the insert direction Ai. The driven rollers 47 to 50 have totally three sets, and provided at intervals of 120° in a circumferential direction of the external cylinder 43. Each of the driven rollers 47 to 50 contacts the inner periphery 40b and rotates corresponding to rotation of the rotating body 40.

Drive rollers 51 and 52 are rotatably attached to the periphery of the inner cylinder 44 along the insert direction Ai. The drive rollers 51 and 52 have totally three sets, and provided at intervals of 120° in a circumferential direction of the internal cylinder 44. The drive rollers 51 and 52 contact the external cylinder 40a.

The drive roller 51 locates between the driven rollers 47 and 48 and clamps the rotating body 40 between the driven rollers 47 and 48. Moreover, the drive roller 52 locates between the driven rollers 49 and 50 and clamps the rotating body 40 at the position between the driven rollers 49 and 50. The drive rollers 51 and 52 are rotated by a transmission gear tube 53 to rotate the rotating body 40.

The internal cylinder 44 is a double cylinder having an inner cylinder 57 and an outer cylinder 59 that is placed outside of the inner cylinder 57. The inner cylinder 57 and the outer cylinder 59 are uniformed through a front end 58 formed in a doughnut shape. A space for disposing the transmission gear tube 53 is provided between the inner cylinder 57 and the outer cylinder 59.

The inner cylinder 57 has inner diameter larger than the outer diameter of the insert section 13 for sufficiently obtaining the space 46. The drive rollers 51 and 52 are rotatably attached to the outer cylinder 59.

The transmission gear tube 53 is fit onto the outer periphery of the inner cylinder 57. Teeth 53a for engaging with the drive rollers 51 and 52 are formed on the outer periphery of the transmission gear tube 53. It is noted that the teeth 53a are formed at two positions corresponding to the drive rollers 51 and 52, but they may be continuously formed. The teeth 53a are worms, so that and the worm wheels are used for the drive rollers 51 and 52.

A gear 54 having a number of teeth in its circumferential direction is formed at the rear end of the outer periphery of the transmission gear tube 53. A pinion 56 provided at a front end of the rotation torque transmission member 55 engages with the gear 54. The pinion 56 is rotated by the rotation torque transmission member 55 and the rotation is transmitted to the transmission gear tube 53 via the gear 54.

It is noted the gear 54 is formed long in the insert direction Ai so as not to release engagement of the gear 54 and the pinion 56 even when the main body 41 moves toward the axis direction of the insert section 13. The rotation torque transmission member 55 and the protective sheath 36 are slidably supported to the support ring 37.

The transmission gear tube 53 is rotated around the central axis of the internal cylinder 44 with driving force from the pinion 56. Thereby, the drive rollers 51 and 52 are rotated, and the rotating body 40 is rotated by the rotation of the drive rollers 51 and 52.

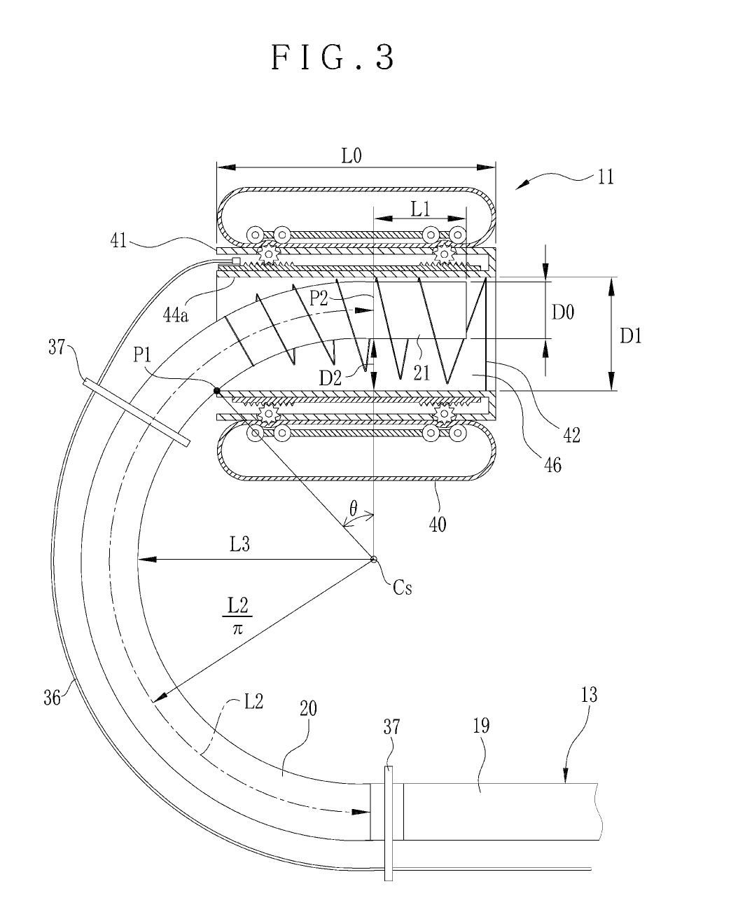

Shown in FIG. 3, L0, L1 and L2 respectively represent lengths of the main body 41, the distal end 21 and bending portion 20. L3 represents the inner diameter of the bending portion 20 bended at 180° and D0 represents the outer diameter of the insert section 13. In this case, the self propelling device 11 and the insert section 13 are formed so as to satisfy the following formula 1 and formula 2;

L0≦L1+L3 [formula 1]

L3=L2/π−D0/2 [formula 2]

Moreover, P1 represents a position where the bending portion 20 contacts an edge of the insertion hole 44a when the bending portion 20 is bended at 180°, and the P2 represents a boundary between the distal portions 21 and the bending portion 20, and Cs represents a center of the bending portion 20. Then, θ represents an angle intersecting a line joining P1 and Cs, and a line joining P2 and Cs. Moreover, D1 represents an inner diameter of the insertion hole 44a and D2 represents size of the space 46. In this case, the self propelling device 11 and the insert section 13 are formed so as to satisfy the following formula 3 and formula 4;

D2=D1−D0=L3(1−cos θ) [formula 3]

θ≈sin⁻¹{(L0−L1)/L3} [formula 4]

To satisfy the following formulas 1 to 4 is a condition for allowing the bending portion 20 to bend at 180°. If the above condition is satisfied, there is no possibility that bending operation of the bending portion 20 is blocked by the self propelling device 11.

Here, the larger the D2 that is a size of the 46, the easier the bending operation of the bending portion 20. However, there is a case where DO that is outer diameter of the insert section 13 in formula 3 can not be small because of the large size of D2. In this case, it is necessary to enlarge the size of D1 that is inner diameter of the insertion hole 44a, which makes difficult to insert the insert section 13 with the self propelling device 11 into the large intestine since the size of the self propelling device 11 becomes large. Therefore, the D2 that is a size of the space 46 is determined considering D0 that is outer diameter of the insert section 13 and the size of the self propelling device 11.

Next, operation of the endoscope system 2 having the above-mentioned constitution is explained. First, the support rings 37 are respectively attached to the predetermined plural positions on the insert section 13. Next, the rotation torque transmission member 55 is inserted through the holes of the support rings 37 via the protective sheath 36. Then, the self propelling device 11 is attached to the bending portion 20 via the coil spring 42.

The large loop 42a is fixed to the inner periphery of the internal cylinder 44 in advance. After the distal portion 21 is inserted through the insertion hole 44a, the small loop 42b is fit onto the outer periphery of the bending portion 20. In particular, the distal portion 21 is inserted in the small loop 42b in a manner that the diameter of the small loop 42b is enlarged against the elastic deformation thereof. When the small loop 42b reaches the predetermined position of the bending portion 20 after passing through the distal portion 21, force applied to the small loop 42b is released. Thereby, the small loop 42b is fixed so as to fasten the predetermined position of the bending portion 20. Here, the attachment of the self propelling device 11 is completed.

After attachment of the self propelling device 11, the processor, light source unit, control device are turned on, then the patients' information are entered in an electronic medical chart server from the predetermined terminal. Next, the insert section 13 with the self propelling device 11 is inserted into the large intestine of the patient.

When the distal portion 21 is advanced front of, for example, the sigmoid colon of the large intestine, the operation unit is operated to turn on electric source of the drive source 22. Then, command for advancing forward is input by button operation of the operation unit. The rotation torque transmission member 55 is rotated in the predetermined direction by the drive source 22, and the transmission gear tube 53 is rotated by rotation of the pinion 56 corresponding to the rotation of the rotation torque transmission member 55.

Rotation of the transmission gear tube 53 is transmitted to the drive rollers 51 and 52, and the drive rollers 51 and 52 rotate the rotating body 40. The rotating body 40 is rotated in a manner to contact the inner wall of the large intestine, to generate driving power in the insert direction Ai. The self propelling device 11 hauls the intestine wall of sigmoid colon with driving power from the rotating body 40 to carry the front end of the insert section 13 forward along the intestine wall of sigmoid colon.

When the command for speed change is input by button operation of the operation unit, the rotation speed of the rotation torque transmission member 55 is changed through the drive source 22. Therefore, moving speed of the self propelling device 11 is changed. Moreover, when the command for carrying backward is input by button operation of the operation unit, the rotation torque transmission member 55 is reversely rotated through the drive source 22 and the front end side of the insert section 13 is carried backward. Furthermore, when the stop command is input through button operation of the operation unit, the drive source 22 stops and the self propelling device 11 also stops. Through the appropriate operation described above, the front end side of the insert section 13 can be carried to the desired position such as back of the sigmoid colon.

The operator operates the angle knob 15 to bend the bending portion 20 and orients the distal portion 21 in a desired direction. Shown in FIG. 3, the space 46 is sufficiently obtained. When the bending portion 20 bends, the bending portion 20 et al. are easy to move in the insertion hole 44a in its radial direction since the fixing position of the large loop 42a and the small loop 42b are separate each other in an axis direction of the internal cylinder 44. Therefore, D2 that is size of the 46 can be maximally obtained. This allows easy bend of the bending portion 20 in the insertion hole 44a. Consequently, bending operation of the bending portion 20 is not block by the self propelling device 11, so that the bending portion 20 can be bended smoothly in a similar manner where the self propelling device 11 is not attached.

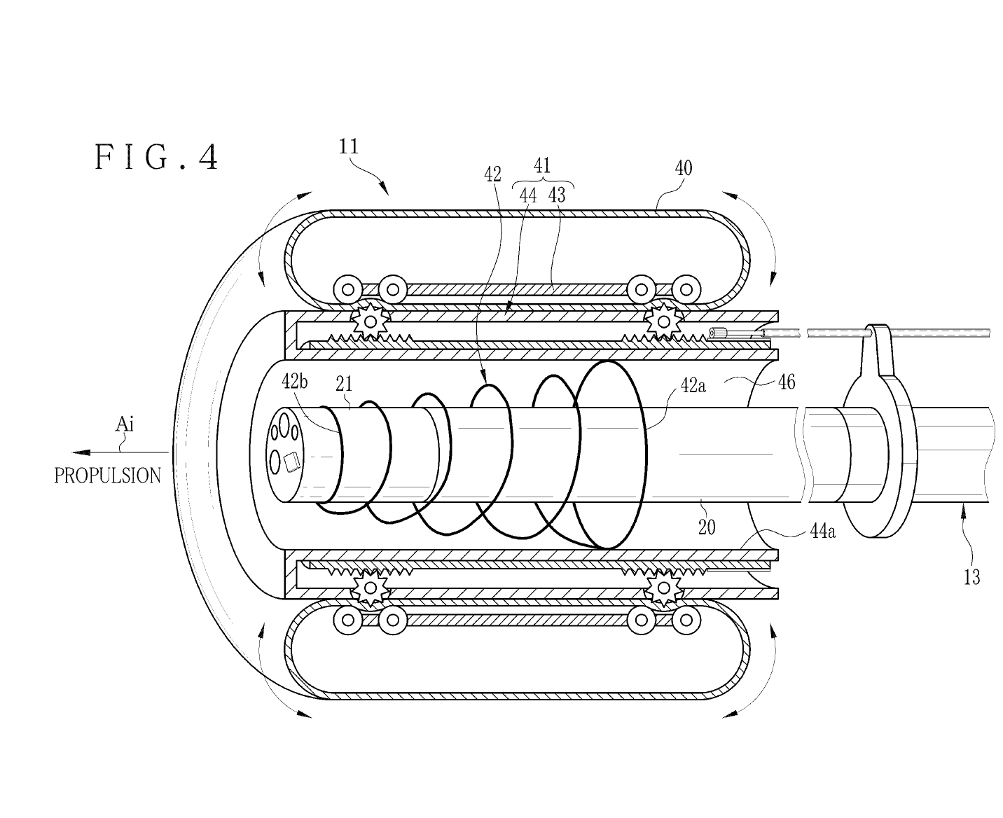

Shown in FIG. 4, it may be possible that the coil spring 42 is reversely disposed in a front-back direction. In particular, the small loop 42b is fixed on the outer periphery of the distal portion 21, and the large loop 42a is fixed on the inner periphery of the internal cylinder 44. In this case, it is preferable that whole circumferences of the large loop 42a and the small loop 42b may be respectively fixed on the inner periphery of the internal cylinder 44 and the outer periphery of the distal portion 21.

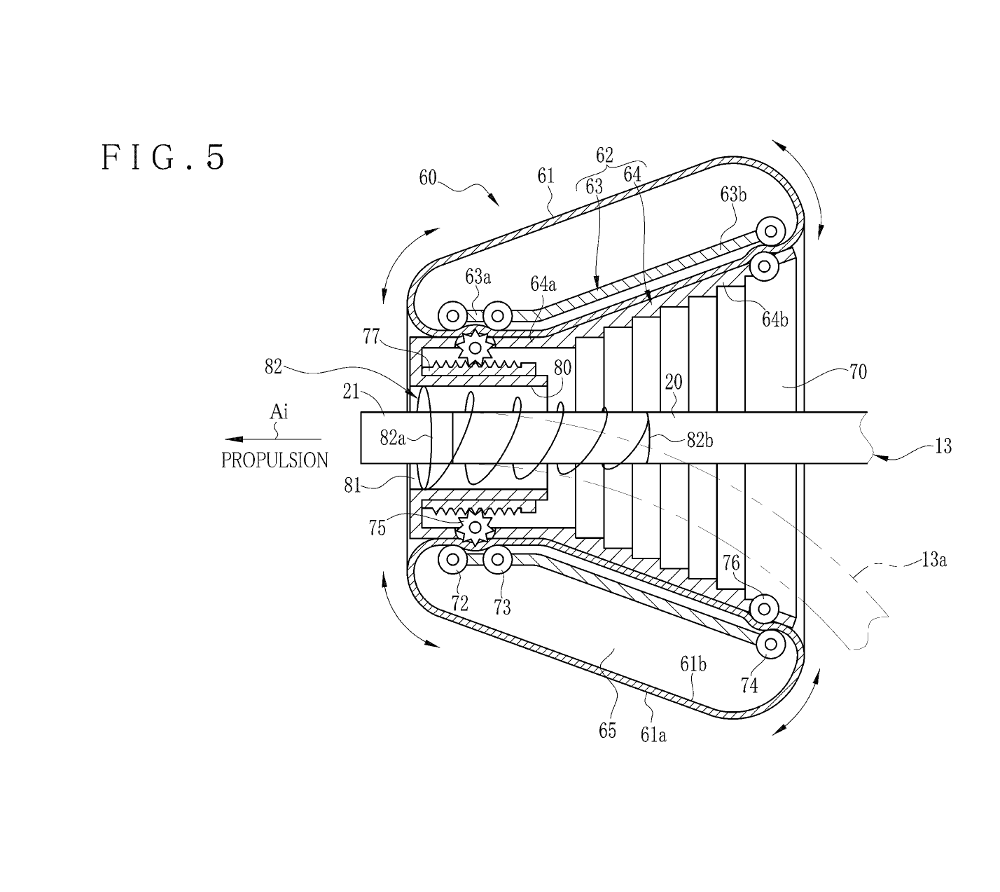

Next, a self propelling device 60 in the second embodiment of the present invention will be described with reference to the FIG. 5. It is noted that the parts having the same structure and function as the above first embodiment have the same reference numerals, and their description will not be repeated.

The self propelling device 60 includes a rotating body 61 and a main body 62. Since the rotating body 61 is basically same as the rotating body 40 of the first embodiment, explanation thereof will not be repeated. The main body 62 includes an external cylinder 63 and an internal cylinder 64. The external cylinder 63 is disposed in a space 65 inside of the rotating body 61. The external cylinder 63 rotatably supports the rotating body 61 in a similar manner to the external cylinder 43 of the first embodiment. The internal cylinder 64 is disposed between the insert section 13 and an outer surface 61a of the rotating body 61.

The external cylinder 63 has a cylinder 63a and a skirt 63b. The internal cylinder 64 has a cylinder 64a and a skirt 64b. The skirts 63b and 64b are respectively connected to the rear end of the cylinders 63a and 64a. The skirts 63b and 64b gradually spread toward the rear end of the insert section 13. Moreover, the inner periphery of the skirts 63b and 64b are formed step-shape. Inner periphery of the skirts 63b and 64b forms a space 70 in the main body 62 for smoothly bending the bending portion 20.

Driven rollers 72, 73 and 74 are rotatably attached to a peripheral surface of the external cylinder 63 along the insertion direction Ai. Each of the driven rollers 72 to 74 has totally three sets, and provided at intervals of 120° in a circumferential direction of the external cylinder 63. The driven 72 is disposed at front end of the cylinder 63a. The driven roller 73 is disposed at rear end of the cylinder 63a. The driven roller 74 is disposed at rear end of the skirt 63b. Each of the driven rollers 72 to 74 contacts an inner surface 61b of the rotating body 61 and rotates according to rotation of the rotating body 61.

A drive roller 75 and a driven roller 76 are rotatably attached to a peripheral surface of the internal cylinder 64 along the insertion direction Ai. Each of the drive roller 75 and the driven roller 76 has totally three sets, and provided at intervals of 120° in a circumferential direction of the internal cylinder 64 and contacts an outer surface 61a. The drive roller 75 is disposed in the middle of the cylinder 64a. The driven roller 76 is disposed at the rear end of the skirt 64b.

The drive roller 75 locates between the driven rollers 72 and 73 and clamps the rotating body 61 between the driven rollers 72 and 73. Moreover, the driven roller 76 clamps the rotating body 61 with the driven roller 74. The drive roller 75 is rotated by a transmission gear tube 77 to rotate the rotating body 61. The driven roller 76 rotates corresponding to the rotation of the rotating body 61.

Since the transmission gear tube 77 is basically same as the transmission gear tube 53 of the first embodiment, explanation thereof will not be repeated. It is noted that the rotation torque transmission member 55 for rotating the transmission gear tube 77 and the pinion 56 are omitted in the drawings.

The cylinder 64a has an insertion hole 80 into which the bending portion 20 et al. are inserted. The inner diameter of the insertion hole 80 is sufficiently larger than the outer diameter of the bending portion 20. Therefore, a space 81 which has sufficient space not to block the bend of the bending portion 20 is provided between the bending portion 20 et al. and the inner periphery of the cylinder 64a forming the insertion hole 80.

The bending portion 20 et al. are supported in the air in the insertion hole 80 by the coil spring formed in truncated cone shape, in a similar manner to the first embodiment. The coil spring 82 has a large loop 82a and a small loop 82b. The large loop 82a is fixed to the inner periphery of the cylinder 64a. The small loop 82b is fixed to the outer periphery of the bending portion 20. Consequently, bending operation of the bending portion 20 can be smoothly carried out in a similar manner to the first embodiment.

Further, in the self propelling device 60, a space 70 formed by the skirt 64b is provided, in addition to the space 81 formed by the insertion hole 80. Therefore, shown with a phantom line 13a, more smooth bending operation of the bending portion 20 can be carried out compared to the first embodiment.

It is noted that the location and the number of the driven roller and the drive roller are not limited to the above first and second embodiments and may be properly changeable.

In the embodiments described above, whole circumferences of the large and small loops of the coil spring are respectively fixed to the internal cylinder or the inner periphery of the internal cylinder, the outer periphery of the bending portion, but the present invention is not limited to this and several points of the large and small loops may be fixed to the inner periphery and the outer periphery.

In the above first embodiment, the large loop 42a is fixed to the front end side or rear end side of the main body 41, and the small loop 42b is detachably fit to the middle portion of the bending portion 20 or the distal portion 21, but the present invention is not limited to this and for example, the large loop 42a may be fixed to the middle portion of the main body 41 and the small loop 42b may be fixed to the middle portion or the rear end of the bending portion 20. Moreover, it may be possible to change length of the coil spring 82 in the second embodiment and appropriately change positions of the large loop 82a and the small loop 82b.

In the above embodiments, the toroid-shaped rotating body is used, but the shape of the rotating body is not limited to this, for example, an endless belt formed in band-shape may be used.

The colonoscope is used in the above embodiments, but the present invention can be applied to a gastroscope and so on. Further, the present invention is not limited for medical diagnosis use, but may be applied to a self propelling device attached to various endoscopes such as industrial endoscope or ultrasonic endoscope.

Various changes and modifications are possible in the present invention and may be understood to be within the present invention.

What is claimed is:

1. A self propelling device attached to a front end side of an insert section of an endoscope and propelling said front end side in a body in an axis direction comprising:
an internal cylinder having an insertion hole into which said front end side is inserted with a predetermined space;
an external cylinder disposed outside of said internal cylinder;
a rotating body wound to surround the outer periphery and inner periphery of said external cylinder;
a driver for rotatably driving said rotating body along an axis direction of said external cylinder; and
a support member provided between an inner periphery of said internal cylinder and an outer periphery of said front end side, for movably supporting said front end side in an at least radial direction in said insertion hole.

2. A self propelling device according to claim 1, said support member includes a first end that is fixed to the inner periphery of said internal cylinder, and includes a second end that is detachably fit on the outer periphery of said front end side, wherein said first end and said second end are separated in said axis direction.

3. A self propelling device according to claim 2, wherein said support member is a coil spring formed in a circular truncated cone shape, said first end is positioned at a bottom having large diameter and said second end is positioned at a top having small diameter.

4. A self propelling device according to claim 2, wherein said second end is fit onto an outer periphery of a bending portion that is bendable and provided at said front end side.

5. A self propelling device according to claim 1, wherein said internal cylinder and said external cylinder are formed in skirt shape gradually spreading toward the rear end side of said insert section.
